(19) United States
(12) Patent Application Publication
(10) Pub. No.: US 2025/0340840 A1
(43) Pub. Date: Nov. 6, 2025

(54) BIOLOGICAL AGE REVERSING PREPARATION AND METHOD FOR PREPARING SAME

(71) Applicant: JIXIAN HONG, SHANGHAI (CN)

(72) Inventors: JIXIAN HONG, SHANGHAI (CN); HUA LIU, SHANGHAI (CN)

(21) Appl. No.: 18/864,901

(22) PCT Filed: May 30, 2022
(86) PCT No.: PCT/CN2022/096096

§ 371 Date: Nov. 12, 2024

(30) Foreign Application Priority Data
May 12, 2022 (CN) 202210514823.0

(51) Int. Cl. C12N 5/0783; A61K 35/17; C12Q 1/68

(52) U.S. Cl. CPC C12N 5/0636; A61K 35/17; C12Q 1/68; C12N 2500/76; C12N 2501/2302; C12N 2501/2306; C12N 2501/2315; C12N 2501/24; C12N 2501/90; C12Q 2600/154

(57) ABSTRACT

A method for preparing a biological age reversing preparation, comprising the steps of: placing cytotoxic T lymphocytes, a cell mitogen, a cytokine, and an immunologic adjuvant in a liquid cell culture medium in a culture container for co-culture to obtain an immune cell culture, and isolating a population of cells with immune activity from the immune cell culture to obtain the biological age reversing preparation. By using the biological age reversing preparation, the biological age can be reversed significantly. The biological age reversing preparation comprising 10¹⁰ cytotoxic T lymphocytes is used three times within a week, resulting 1.3 years of biological age reversal after the preparation is continuously used for 3 months, and 4.5 years of biological age reversal after the preparation is continuously used for 9 months.

### FIELD OF THE DISCLOSURE

The invention relates to the technical field of biological preparation, in particular to a biological age reversing preparation and a method for preparing the same.

### BACKGROUND OF THE DISCLOSURE

Currently, the combination of human growth hormone (GH) or/and GH releasing agents, dehydroepiandrosterone (DHEA), and metformin, can regenerate the thymus in the elderly to prevent age-related immune dysfunction (immunosenescence) or restore immune function (immunosenescence reversal) in the elderly, which is defined as the reversal of epigenetic biological age (biological age) as tested by using gene chips. However, long-term use of such combination may result in toxicity and other possible side effects in humans.

### SUMMARY OF THE DISCLOSURE

The object of the present invention is to provide a biological age reversal preparation and a method for preparing the same, aiming at the deficiency in the prior art.

In order to realize the above object, the technical proposal adopted by the invention is as follows.

A first aspect of the present invention is to provide a method for preparing a biological age reversing preparation comprising the steps of: co-culturing cytotoxic T lymphocytes with a cell mitogen, a cytokine and an immunologic adjuvant in a liquid cell culture medium in a culture vessel to obtain an immune cell culture, and isolating a cell population that is immunocompetent from the immune cell culture to obtain the biological age reversing preparation.

Preferably, a concentration of the cytotoxic T lymphocytes is 1×10³ cells/mL to 1×10¹¹ cells/mL.

Preferably, the cell mitogen is selected from at least one of concanavalin, phytohemagglutinin, pokeweed, lipopolysaccharide, and dextran.

Preferably, a concentration of the cell mitogen is 100,000 units/L to 10 million units/L.

Preferably, the concentration of the cell mitogen is 0.1 mg/L to 10 mg/L.

Preferably, the cytokine is selected from at least one of lymphokine, monokine, a cytokine that activates inflammation, and a cytokine that stimulates hematopoiesis. The lymphokine is derived from lymphocytes, monocytes or lymphokine-producing cells.

Preferably, the cytokine is selected from at least one of interleukin, interferon, colony stimulating factor, chemokine, and transforming growth factor.

Preferably, the cytokine is selected from at least one of interleukin-2, interleukin-6, interleukin-15, and interferon.

Preferably, a concentration of the cytokine is 200,000 units/L to 5,000,000 units/L.

Preferably, the immunologic adjuvant is selected from at least one of a biological adjuvant, an inorganic adjuvant, an organic adjuvant, a synthetic adjuvant, an oil agent, and a Freund's adjuvant.

Preferably, a concentration of the immunologic adjuvant is 0.01 mL/L to 1 mL/L.

Preferably, the culture vessel is a 3D large-volume high-density cell culture vessel.

Preferably, the co-culturing is performed for a period of 3 days to 180 days.

Preferably, the step further comprises: using the immune cell culture or the cell population as a raw material to carry out cloning in the liquid cell culture medium to, obtain a cell line that is immunocompetent.

Preferably, the cloning is selected from any one of an intermittent cyclic stimulation method or a continuous stimulation method.

A second aspect of the present invention is to provide a biological age reversing preparation prepared by the method described above.

A third aspect of the present invention is to provide a method for detecting a change in an expression level of gene methylation by the biological age reversing preparation described above, and the method comprises: using a gene methylation chip for detection.

Compared with the prior art, the invention adopts the technical proposal described above and has the following technical effects. By using the biological age reversing preparation of the present invention, the biological age can be obviously reversed. The biological age reversing preparation including 10¹⁰ cytotoxic T lymphocytes is used three times in one week, and the biological age reversal is 1.3 years after continuous use for 3 months, and 4.5 years after continuous use for 9 months.

### DETAILED DESCRIPTION OF THE EXEMPLARY EMBODIMENTS

The following will be combined with the embodiments of the present invention to clearly and completely describe the technical solutions in the embodiments of the present invention. Obviously, the implementation cases described are only part of the embodiments of the present invention, not all of the embodiments. Based on the implementation capabilities of the present invention, the ordinary technicians in this field have obtained it without any creative work. All other implementation capabilities belong to the scope of protection of the present invention.

It should be noted that without conflict embodiments and features in embodiments of the present invention may combine with each other.

The present invention is further described in connection with a specific embodiment, but it is not a limitation of the present invention.

### Embodiment 1

The present embodiment provides a method for preparing a biological age reversing preparation, comprising the following step.

Co-culturing cytotoxic T lymphocytes with concanavalin, interleukin-2, and 5% tween-80 in a liquid cell culture medium in a 3D large-volume high-density cell culture vessel for 30 days to obtain an immunity cell culture and isolating a cell population that is immunocompetent from the immune cell culture to obtain the biological age reversing preparation.

Wherein a concentration of concanavalin in the liquid cell culture medium is 100,000 units/L; a concentration of interleukin-2 in the liquid cell culture medium is 500,000 units/L; and a concentration of 5% Tween-80 in the liquid cell culture medium is 0.5 mL/L.

### Embodiment 2

Co-culturing cytotoxic T lymphocytes with phytohemagglutinin, interferon, and 5% Tween-80 in a liquid cell culture medium in a 3D large-volume high-density cell culture vessel for 30 days to obtain an immune cell culture, and isolating a cell population that is immunocompetent from the immune cell culture to obtain the reverse biological age preparations

Wherein a concentration of phytohemagglutinin in the liquid cell culture medium is 0.5 mg/L; a concentration of interferon in the liquid cell culture medium is 5 million units/L; and a concentration of 5% Tween-80 in the liquid cell culture medium is 0.1 mL/L.

### Embodiment 3

Co-culturing cytotoxic T lymphocytes with concanavalin, interleukin-2, and 5% tween-80 in a liquid cell culture medium in a 3D large-volume high-density cell culture vessel for 45 days to obtain an immune cell culture, and isolating a cell population that is immunocompetent from the immune cell culture to obtain the biological age reversing preparation.

Wherein a concentration of concanavalin in the liquid cell culture medium is 1 million units/L; a concentration of interleukin-2 in the liquid cell culture medium is 1 million units/L; and a concentration of 5% Tween-80 in the liquid cell culture medium is 0.1 mL/L.

### Embodiment 4

Co-culturing cytotoxic T lymphocytes with phytohemagglutinin, interferon, and 5% tween-80 in a liquid cell culture medium in a 3D large-volume high-density cell culture vessel for 60 days to obtain an immune cell culture, and isolating a cell population that is immunocompetent from the immune cell culture to obtain the biological age reversing preparation.

Wherein a concentration of phytohemagglutinin in the liquid cell culture medium is 1 mg/L; a concentration of interferon in the liquid cell culture medium is 3 million units/L; and a concentration of 5% Tween-80 in the liquid cell culture medium is 0.5 mL/L.

### Test Embodiment

With the increase of age, the number of gene copies gradually increases, the change of gene methylation level also increases, and the deviation of expression results increases. Based on this, gene chip illumina 850K can be used to test the changes of gene methylation with aging.

The biological age reversing preparation containing 10¹⁰ cytotoxic T lymphocytes was applied three times in one week for 3 months. Gene methylation changes were tested by using gene chip illumina 850K, and the biological age obtained by a nonlinear regression analysis was 66;

After continuous use for another 6 months, the methylation changes of more than 800,000 genes were tested by using gene chip illumina 850K. Some data are shown in the table below.

TABLE 1

| gene locus | Gene methylation data |
|---|---|
| cg07881041 | 0.85625064 |
| cg03513874 | 0.868243728 |
| cg05451842 | 0.056959834 |
| eg14797042 | 0.915575221 |
| cg09838562 | 0.067132116 |
| cg09261072 | 0.610496782 |
| cg02404579 | 0.797817048 |
| cg04118974 | 0.63918996 |
| cg01236347 | 0.646615253 |
| cg22585117 | 0.825051055 |
| cg25552317 | 0.809376928 |
| cg23875663 | 0.560348784 |
| cg07659892 | 0.260691538 |
| cg15995909 | 0.899773585 |
| cg23728960 | 0.400339431 |
| cg11993619 | 0.610987544 |
| cg01925883 | 0.649522636 |
| cg03452160 | 0.557117567 |
| cg09430819 | 0.613257422 |
| cg21784030 | 0.638569207 |
| cg13871826 | 0.740332874 |
| cg16474293 | 0.818181818 |
| cg14463239 | 0.621192804 |
| cg05301794 | 0.84797891 |
| cg08829765 | 0.064378645 |
| cg10855276 | 0.863353334 |
| eg12408992 | 0.785294118 |
| cg14370485 | 0.057539347 |
| cg05493344 | 0.696181516 |
| cg05650511 | 0.573154599 |
| cg05530295 | 0.631890661 |
| cg10136773 | 0.897720545 |
| cg20725941 | 0.074437182 |

The biological ages were obtained by the nonlinear regression analysis of the data shown in Table 1 are shown in Table 2.

TABLE 2

| Date of birth | 1954 May |
|---|---|
| Test Year | 2021 |
| Biological age (Horvath algorithm) | 62.9 |
| Biological age (Huannum algorithm) | 54 |
| Epigenetic biological age | 52.5 |
| Theoretical epigenetic biological age | 55.4 |
| Biological age of skin and blood | 62.6 |
| Biological age | 62.2 |
| CD8 lymphoid T cells | 0.08 |
| CD4 lymphoid T cells | 0.18 |
| NK cells | 0.147 |
| B cells | 0.073 |

Because Horvath algorithm is recognized as the most accurate in the world at present, the biological age obtained by Horvath algorithm is used to calculate the biological age.

In summary, the assay by using the chip illumina 850K and ISCAN instrument showed that the biological age reversing preparation containing 10¹⁰ cytotoxic T lymphocyte cells was used three times in one week, and the biological age reversal was 1.3 years after 3 months of continuous use and 4.5 years after 9 months of continuous use.

The foregoing is only a preferred embodiment of the present invention, and it does not thus limit the embodiment and scope of protection of the present invention. It should be appreciated to those skilled in the art that all schemes resulting from equivalent substitutions and obvious variations made using the contents of the present specification should be covered by the scope of protection of the present invention.

1. A method for preparing a biological age reversing preparation, characterized in that the method comprises the following step:
co-culturing cytotoxic T lymphocytes with a cell mitogen, a cytokine and an immunologic adjuvant in a liquid cell culture medium in a culture vessel to obtain an immune cell culture, and isolating a cell population that is immunocompetent from the immune cell culture to obtain the biological age reversing preparation.

2. The method according to claim 1, characterized in that the cell mitogen is selected from at least one of concanavalin, phytohemagglutinin, pokeweed, lipopolysaccharide, and dextran.

3. The method according to claim 1, characterized in that the cytokine is selected from at least one of lymphokine, monokine, a cytokine that activates inflammation, and a cytokine that stimulates hematopoiesis; wherein the lymphokine is derived from lymphocytes, monocytes or lymphokine-producing cells.

4. The method according to claim 3, characterized in that the cytokine is selected from at least one of interleukin, interferon, colony stimulating factor, chemokine, and transforming growth factor.

5. The method according to claim 4, characterized in that the cytokine is selected from at least one of interleukin-2, interleukin-6, interleukin-15 and interferon.

6. The method according to claim 1, characterized in that the immunological adjuvant is selected from at least one of a biological adjuvant, an inorganic adjuvant, an organic adjuvant, a synthetic adjuvant, an oil agent and a Freund's adjuvant.

7. The method according to claim 1, characterized in that the step further comprises: using the immune cell culture or the cell population as a raw material to carry out cloning in the liquid cell culture medium, to obtain a cell line that is immunocompetent.

8. The method according to claim 7, characterized in that the cloning is selected from any one of an intermittent cyclic stimulation method or a continuous stimulation method.

9. A biological age reversing preparation prepared by a method as claimed in claim 1.

10. A method for detecting a change in an expression level of gene methylation by the biological age reversing preparation as claimed in claim 9, the method comprising: using a gene methylation chip for detection.

11. A biological age reversing preparation prepared by a method as claimed in claim 2.

12. A method for detecting a change in an expression level of gene methylation by the biological age reversing preparation as claimed in claim 11, the method comprising: using a gene methylation chip for detection.

13. A biological age reversing preparation prepared by a method as claimed in claim 3.

14. A method for detecting a change in an expression level of gene methylation by the biological age reversing preparation as claimed in claim 13, the method comprising: using a gene methylation chip for detection.

15. A biological age reversing preparation prepared by a method as claimed in claim 4.

16. A method for detecting a change in an expression level of gene methylation by the biological age reversing preparation as claimed in claim 15, the method comprising: using a gene methylation chip for detection.

17. A biological age reversing preparation prepared by a method as claimed in claim 5.

18. A method for detecting a change in an expression level of gene methylation by the biological age reversing preparation as claimed in claim 17, the method comprising: using a gene methylation chip for detection.

19. A biological age reversing preparation prepared by a method as claimed in claim

6.

20. A method for detecting a change in an expression level of gene methylation by the biological age reversing preparation as claimed in claim 19, the method comprising: using a gene methylation chip for detection.

21. A biological age reversing preparation prepared by a method as claimed in claim

7.

22. A method for detecting a change in an expression level of gene methylation by the biological age reversing preparation as claimed in claim 21, the method comprising: using a gene methylation chip for detection.

23. A biological age reversing preparation prepared by a method as claimed in claim 8.

24. A method for detecting a change in an expression level of gene methylation by the biological age reversing preparation as claimed in claim 24, the method comprising: using a gene methylation chip for detection.
